(19) United States
(12) Patent Application Publication
(10) Pub. No.: US 2002/0051791 A1
(43) Pub. Date: May 2, 2002

(54) Methods for protection against lethal infection with bacillus anthracis

(76) Inventors: Darrel R. Galloway, Dublin, OH (US); Alfred J. Mateczun, Albuquerque, NM (US)

Correspondence Address: NAVAL MEDICAL RESEARCH CENTER, ATTN: (CODE 00L), 503 ROBERT GRANT AVENUE, SILVER SPRING, MD, 20910-7500, US

(21) Appl. No.: 09/747,521

(22) Filed: Dec. 21, 2000

Related U.S. Application Data
(60) Provisional application No. 60/171,459, filed on Dec. 22, 1999.

(51) Int. Cl. A61K 48/00; A61K 39/07

(52) U.S. Cl. 424/190.1; 514/44

(57) ABSTRACT

Methods of intducing an immune response which protects a susceptible animal subject from lethal infection with Bacillus anthracis (B. anthracis) are provided. One method comprises administering B. anthracis lethal factor (LF) or an immunogenic fragment thereof to the subject. A second method comprises administering LF or an immunogenic fragment thereof and the B anthracis protective antigen (PA) to the subject. A third method comprises administering a polynucleotide which encodes B. anthracis LF or an immunogenic fragment thereof to the subject. A fourth method comprises administering a polynucleotide which encodes LF or an immunogenic fragment thereof and a polynucleotide which encodes the B. anthracis PA to the subject. The present invention also relates to a protein or peptide based-immunogenic composition for preparing a vaccine which is capable of prophylactically protecting a subject against lethal effects of infection with B. anthracis.

This application claims priority from U.S. Provisional Application Serial No. 60/171,459 filed Dec. 22, 1999.

### BACKGROUND OF THE INVENTION

Anthrax is a disease caused by the spore-forming bacterium, Bacillus anthracis. A bacterium that is readily found in soil, B. anthracis primarily causes disease in plant-eating animals. Anthrax infection of humans is infrequent (1 in 100,000). When humans do become infected, they usually acquire the bacterium from contact with infected animals, animal hides or hair, or animal feces. The human disease has a relatively short incubation period (less than a week) and usually progresses rapidly to a fatal outcome.

In humans, anthrax can occur in three different forms: cutaneous anthrax, gastrointestinal anthrax and inhalation anthrax. Cutaneous anthrax, the most common form in humans, is usually acquired when the bacterium, or spores of the bacterium, enter the body through an abrasion or cut on the skin. The bacteria multiply at the site of the abrasion, cause a local edema, and a series of skin lesions—papule, vesicle, pustule and necrotic ulcer—are sequentially produced. Lymph nodes nearby the site are eventually infected by the bacteria and, in cases where the organisms then enter the bloodstream (20% of cases), the disease is often fatal.

Gastrointestinal anthrax is caused by eating contaminated meat. Initial symptoms include nausea, vomiting and fever. Later, infected individuals present with abdominal pain, severe diarrhea and vomiting of blood. This type of anthrax is fatal in 25% to 60% of cases.

Inhalation anthrax (also called woolsorters' disease) is acquired through inhalation of the bacteria or spores. Initial symptoms are similar to those of a common cold. Symptoms then worsen and these individuals present with high fever, chest pain and breathing problems. The infection normally progresses systemically and produces a hemorrhagic pathology. Inhalation anthrax is fatal in almost 100% of cases.

### Virulence Determinants of Anthrax Bacillus

B. anthracis possesses two major virulence components. The first virulence component is a polysaccharide capsule which contains poly-D-glutamate polypeptide. The poly-D-glutamate capsule is not itself toxic but plays an important role in protecting the bacterium against anti-bacterial components of serum and phagocytic engulfment. The poly-D-glutamate capsule, therefore, enables the B. anthracis bacterium to withstand non-specific immunity of the human host and multiply therein.

As the B. anthracis bacterium multiplies in the host, it produces a secreted toxin which is the second virulence component of the organism. This anthrax toxin mediates symptoms of the disease in humans. The anthrax toxin is comprised of three distinct proteins encoded by the bacterium, called protective antigen (PA), lethal factor (LF) and edema factor (EF). PA is the component of the anthrax toxin that binds to host cells using an unidentified cell-surface receptor. Once it binds to cell surfaces, EF or LF can subsequently interact with the bound PA. The complexes are then internalized by the host cell with significant effects. EF is an adenylate cyclase which causes deregulation of cellular physiology, resulting in edema. LF is a metalloprotease that cleaves specific signal transduction molecules within the cell (MAP kinase kinase isoforms), causing deregulation of said pathways, and cell death. Injection of PA, LF or EF alone, or LF in combination with EF, into experimental animals produces no effects. However, injection of PA plus EF produces edema. Injection of PA plus LF is lethal, as is injection of PA plus EF plus LF.

### Anthrax Vaccines

The present anthrax vaccine, which was developed during the 1950s and 1960s, is prepared from the supernatant of the V770-NP1-R strain of B. anthracis. The vaccine consists primarily of the PA antigen adsorbed onto aluminum hydroxide, although the precise composition of the vaccine is undetermined. The vaccine is effective as shown by survival of vaccinated monkeys that were challenged with airborne B. anthracis spores. A retrospective analysis of the anthrax vaccine showed 93% fewer anthrax infections among vaccinated people, compared to unvaccinated people.

Although the traditional anthrax vaccine is effective, it has a number of shortcomings. For example. it requires multiple administrations, plus annual boosters, for maximum effectiveness. Typically, the existing anthrax vaccine is given in a series of six doses over an 18 month. The first vaccination of the series must be given at least four weeks before exposure to the disease. Subsequent to the six-dose series, yearly boosters are required to retain protective immunity. In addition, the specific composition of the vaccine has not been determined and may vary from lot-to-lot. Finally, the vaccine causes adverse reactions in some people who receive it.

Accordingly, it is desirable to have additional compositions which offer prophylactic protection against a lethal Bacillus anthracis infection.

### Summary of the Invention

The present invention provides methods of inducing an immune response which protects an animal subject from lethal infection with Bacillus anthracis (B. anthracis). One method comprises administering an effective amount of wild-type, or preferably a mutated form of, B. anthracis lethal factor (LF) or an immunogenic fragment thereof to the subject. In one embodiment the LF protein comprises the amino acid sequence, SEQ ID NO.2 shown in FIG. 1. In one embodiment the LF fragment comprises amino acid 9 through amino acid 252 of the sequence, SEQ ID NO:2, shown in FIG. 1. A second method comprises administering an effective amount of a mutated LF protein or a fragment thereof and an effective amount of the B anthracis protective antigen (PA) or an immunogic fragment of the PA protein to the subject. In one embodiment, the immunogenic fragment of the B anthracis protective antigen comprises consecutively amino acid 175 through amino acid 735 of the amino acid sequence, SEQ. ID NO: 4, shown in FIG. 2. A third method comprises administering a polynucleotide or nucleic acid comprising a sequence encoding B. anthracis LF protein or a fragment thereof to the subject. In one embodiment the polynucleotide which encodes the full-length mature LF protein comprises consecutively nucleotide 100 through nucleotide 2430 of the sequence, SEQ ID NO.1, shown in FIG. 1. In one embodiment the polynucleotide which encodes an LF fragment comprises consecutively nucleotide 125 through nucleotide 855 of the sequence, SEQ ID NO:1, shown in FIG. 1. A fourth method comprises administering a polynucleotide which comprises a coding sequence for a mutated LF protein or immunogenic fragment thereof and a polynucleotide which comprises a coding sequence for the B. anthracis PA protein or an immunogenic fragment thereof to the subject. In one embodiment, the nucleotide sequence encoding the full-length, mature PA protein comprises consecutively nucleotide 88 through nucleotide 2295 of the sequence, SEQ. ID NO:3, shown in FIG. 2. In one embodiment, the nucleotide sequence which encodes an immunogenic fragment of the PA protein, comprises consecutively nucleotide 610 through nucleotide 2295 of the sequence, SEQ ID NO: 3, shown in FIG. 2. The present methods stimulate or increase the level of antibodies which inactivate the B. anthracis lethal toxin in the subject.

The present invention also relates to a protein or peptide based-immunogenic composition for preparing a vaccine which is capable of prophylactically protecting a subject against lethal effects of infection with B. anthracis or exposure to a toxic agent which is produced by B. anthracis. The protein or peptide based immunogenic composition comprises a purified or recombinant LF protein or immunogenic fragment thereof and a purified or recombinant PA protein or immunogenic fragment thereof. The present invention also relates to a nucleic acid-based immunogenic composition comprising a nucleic acid which comprises a sequence encoding the LF protein or an immunogenic fragment thereof and a polynucleotide which comprises a sequence encoding the PA protein or an immunogenic fragment thereof.

### BRIEF DESCRIPTION OF THE FIGURES

FIG. 1 shows a nucleotide sequence, SEQ ID NO:1, of a DNA which encodes wild-type B. anthracis protein and the amino acid sequence, SEQ ID NO. 2, derived therefrom.

FIG. 2 shows a nucleotide sequence, SEQ ID NO.3, of a DNA which encodes a wild-type B. anthracis PA and the amino acid sequence, SEQ ID NO.4, of the protein derived therefrom.

FIG. 3 shows the Plasmid pCI (Promega Inc.), the eucaryotic expression vector which was used to express aa 9-252 of B. anthracis lethal factor protein and aa 175 -735 of B. anthracis protective antigen protein.

FIG. 4 is a bar graph showing the serum antibody titers in BALB/c mice immunized with pCPA, pCLF4, or a combination of pCPA and pCLF4 against purified lethal factor protein (A) or protective antigen (B).

FIG. 5 is a bar graph showing the serum antibody titers in BALB/c mice immunized against

(A) protective antigen with pCPA, pCPA and pCLF4, and pCPA and pCLF4 boosted with protective antigen (PA) and mutant lethal factor protein (LF7) on day 28.

(B) lethal factor with pCLF4, pCLF4 and pCPA, and pCPA and pCLF4 boosted with protective antigen (PA) and mutant lethal factor protein (LF7) on day 28.

FIG. 6 is a graph showing the neutralization of anthrax toxin by rabbit anti-LF4 antibody. Various dilutions of anti-LF4 serum were pre-incubated with rLF (●) for 1 h. The mixture was added to J774A.1 cells in the presence of Letx for 7 h and cell viability was measured. Absence of MTT (▪). Negative Letx control ( ).

### DETAILED DESCRIPTION OF THE INVENTION

The present invention relates to immunogenic compositions and methods which use such immunogenic compositions to prophylactically protect an animal subject against a lethal infection with B. anthracis. In accordance with the present invention, Applicants have shown that immunogenic compositions that comprise a nucleic acid which encodes B. anthracis LF or fragment thereof either alone or in combination with a nucleic acid that encodes B. anthracis PA or a fragment thereof are capable of inducing production of enhanced levels of antibodies which inactivate the B. anthracis lethal toxin. Applicants have also determined that immunization of animal subjects with such nucleic-acid based compositions protect the animal subjects from a lethal infection with B. anthracis spores.

All references cited herein are specifically incorporated herein in their entirety.

Peptide-Based Immunogenic Compositions

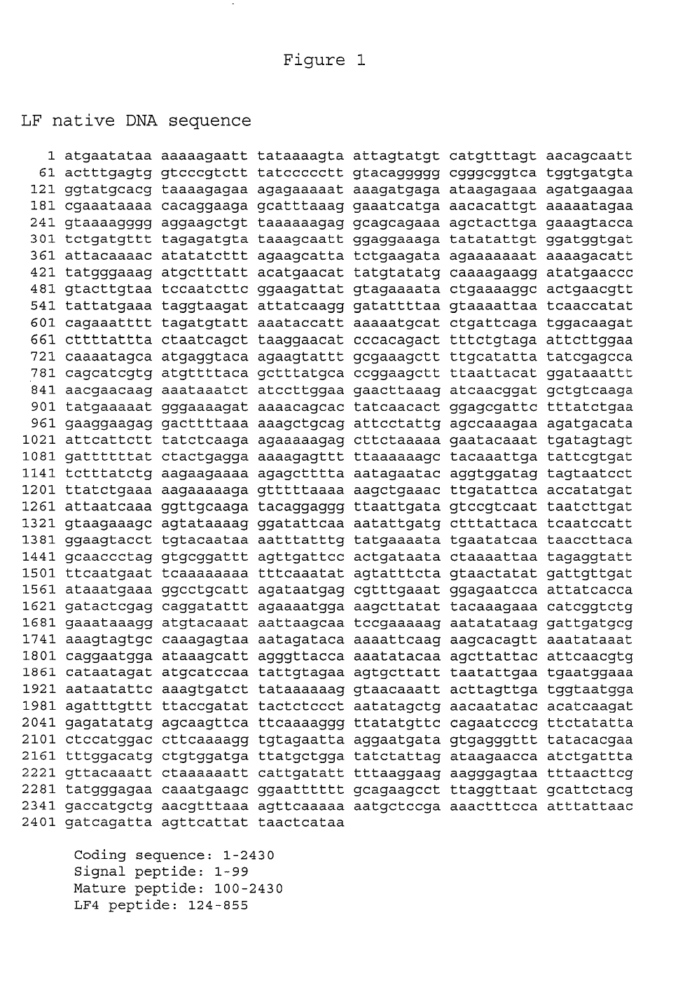

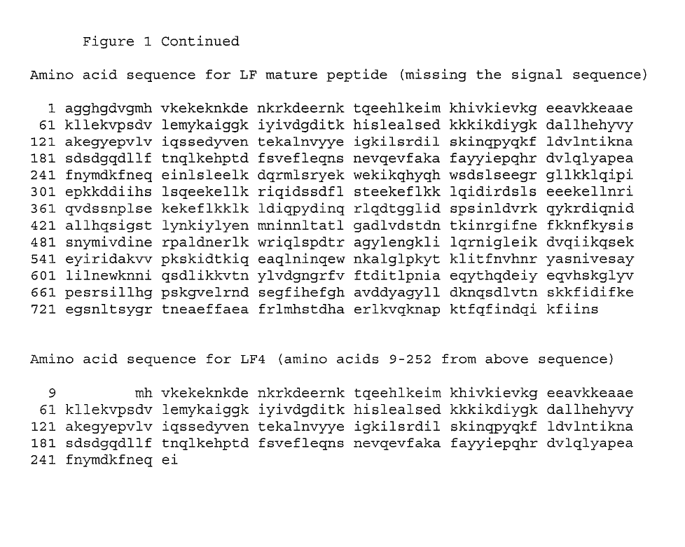

In one aspect, the immunogenic composition comprises a protein or polypeptide which comprises the B. anthracis lethal factor protein, preferably a mutated form of the lethal factor protein such as LF7, which contains a single amino acid substitution of a glutamic acid for a cepteine redidue at position 687, or an immunogenic fragment thereof. As used herein the term “immunogenic fragment” refers to a peptide which is at least 6 amino acids in length, preferably at least 15 amino acids in length, and has the ability to elicit production of antibodies that bind to the wild-type protein from which it was derived, in this case the LF protein. The LF protein may be a full-length, wild-type, mature LF protein. The full-length, wild-type, mature LF protein has a molecular weight of 90 kDa and comprises 764 amino acids. In one embodiment, the full-length, wild-type, mature LF protein comprises the amino acid sequence, SEQ ID NO: 2, shown if FIG. 1. The term “LF protein”, as used herein, also encompasses naturally-occurring and mutated LF proteins whose sequence differs from the sequence shown in FIG. 1. Such variant proteins have an amino acid sequence which is at least 90% identical, preferably at least 95% identical to the amino acid sequence, referred to hereinafter as the “LF protein reference sequence” shown in FIG. 1. Such variant proteins have an altered sequence in which one or more of the amino acids in the LF protein reference sequence is substituted, or in which one or more amino acids are deleted from or added to such sequence. Such variants, when injected into an animal, elicit production of antibodies that bind to the mature, wild-type LF protein, i.e., the LF protein whose sequence is depicted in FIG. 1.

While it is possible to have nonconservative amino acid substitutions, it is preferred that the substitutions be conservative amino acid substitutions, in which the substituted amino acid has similar structural or chemical properties with the corresponding amino acid in the reference sequence. By way of example, conservative amino acid substitutions involve substitution of one aliphatic or hydrophobic amino acid, e.g. alanine, valine, leucine and isoleucine, with another; substitution of one hydroxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

Variant sequences, which are at least 90% identical, have no more than 1 alteration, i.e., any combination of deletions, additions or substitutions, per 10 amino acids of the flanking amino acid sequence. Percent identity is determined by comparing the amino acid sequence of the variant with the reference sequence using MEGALIGN module in the DNA STAR program. One example of a suitable variant of the LF protein shown in FIG. 1 is the LF7 protein which except for a substitution of a glutamic acid for a cysteine at amino acid position 687, has a sequence which is identical to the LF protein reference sequence.

In one embodiment the LF protein immunogenic fragment comprises amino acid 9 through amino acid 252 of the amino acid sequence, SEQ ID NO: 2, shown if FIG. 1. The term LF protein fragment, as used herein, also encompasses LF protein fragments whose sequence differs from the sequence shown in FIG. 1. Such polypeptides have an amino acid sequence which is at least 90% identical, preferably at least 95% identical to the amino acid sequence, referred to hereinafter as the “LF protein fragment reference sequence”, which begins with amino acid 9 and extends through amino acid 252 of the sequence shown in FIG. 1. Such variants, when injected into an animal, elicit production of antibodies that bind to the mature wild-type LF protein, i.e., the LF protein whose sequence is depicted in FIG. 1.

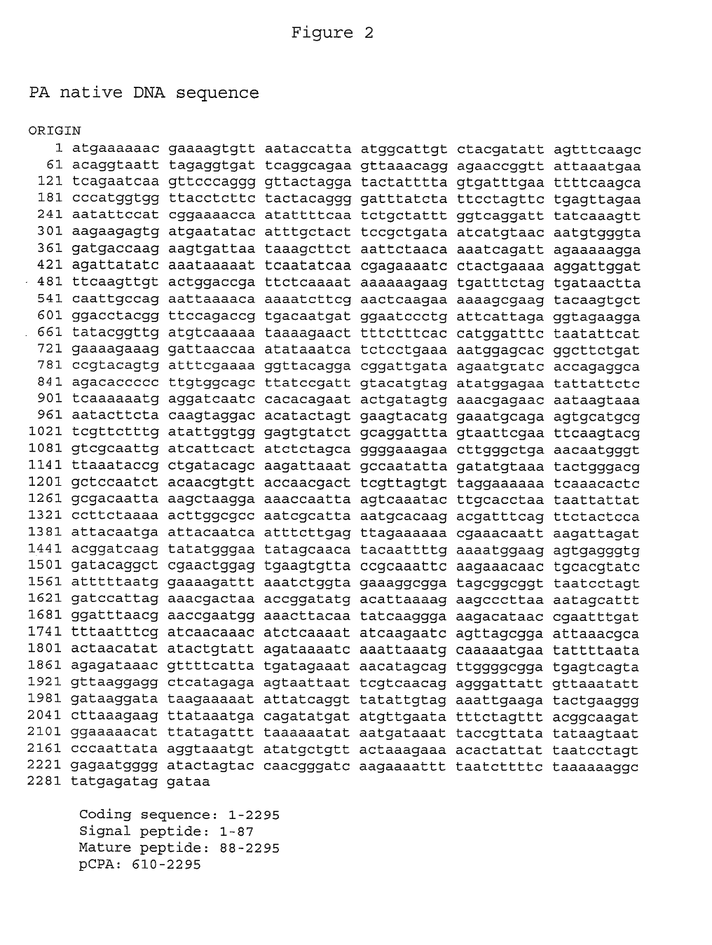

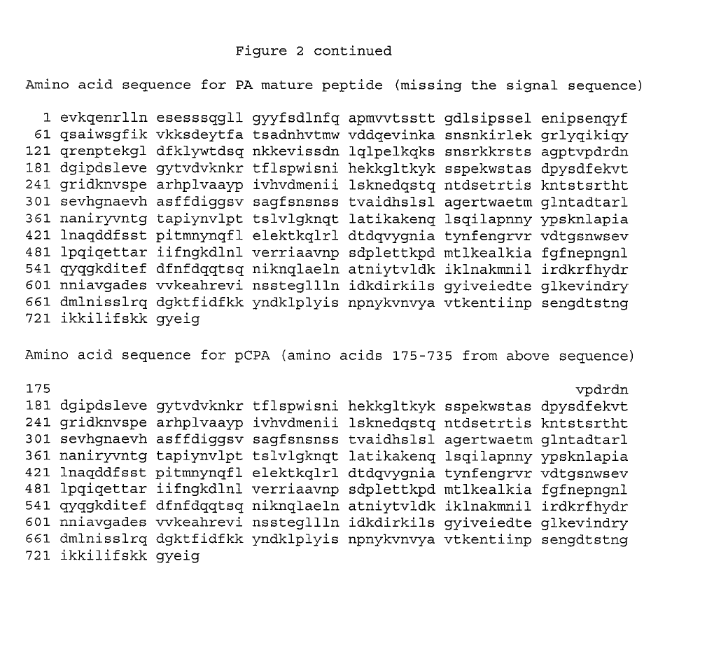

In another aspect, the peptide-based immunogenic composition comprises a mutated LF protein or immunogenic fragment of LF protein and the B. anthracis PA protein or an immunogenic fragment thereof. The full-length, wild-type PA protein has a molecular weight of 83 kDA and comprises 735 amino acids. In one embodiment, the full-length, wild-type, mature PA protein comprises the amino acid sequence, SEQ ID NO: 4, shown if FIG. 2. The term PA protein, as used herein also encompasses wild-type and mutated PA proteins whose sequence differs slightly from the sequence shown in FIG. 2. Such variants have an amino acid sequence which is at least 90% identical, preferably at least 95% identical to the amino acid sequence, referred to hereinafter as the “PA protein reference sequence” shown in FIG. 2. Suitable variants elicit production of antibodies that bind to the wild-type PA protein, i.e., the PA protein whose sequence is shown in FIG. 2.

In one embodiment the PA protein fragment comprises amino acid 175 through amino acid 735 of the amino acid sequence, SEQ ID NO: 4, shown in FIG. 2. The term PA protein fragment, as used herein, also encompasses proteins whose sequence differs slightly from the sequence shown in FIG. 1. Such variants have an amino acid sequence which is at least 90% identical, preferably at least 95% identical to the amino acid sequence, referred to hereinafter as the “PA protein fragment reference sequence”, which begins with amino acid 175 and extends through amino acid 735 of the sequence shown in FIG. 2. Suitable variants of the PA fragment elicit production of antibodies that bind to the wild-type PA protein, i.e. the PA protein whose sequence is shown in FIG. 2.

Methods of Preparing the LF Protein, the PA Protein, and Fragments Thereof

The LF and PA proteins are purified or, preferably, recombinant proteins. Within the context of this application, “purified” PA and LF proteins refers to preparations that are comprised of at least 90% PA or LF, and no more than 10% of the other proteins found in the cell-free extracts or extracellular medium from which these proteins are isolated. Such preparations are said to be at least 90% pure. The LF protein and PA protein may be isolated and purified from the supernatant of B. anthracis using techniques known in the art. One method of isolating the PA protein is described in Methods Enzymol. 165: 103-116, 1988 which is specifically incorporated herein by reference. One method of isolating the LF protein is described in Protein Expression and Purification 18: 293-302, 2000 which is specifically incorporated herein by reference.

Preferably the LF protein, PA protein, and fragments there of are prepared using recombinant techniques. Such techniques employ nucleic acid molecules which encode the LF protein, the PA protein, or fragments thereof. For example, the proteins or fragments thereof may be produced using cell-free translation systems and RNA molecules derived from DNA constructs that encode the such proteins or fragments. Alternatively, the proteins or fragments may be made by transfecting host cells with expression vectors that comprise a DNA sequence that encodes one of the proteins or fragments and then inducing expression of the protein or fragment thereof in the host cells. For recombinant production, recombinant constructs comprising one or more of the sequences which encode the desired protein or fragment are introduced into host cells by conventional methods such as calcium phosphate transfection, DEAE-dextran mediated transfection, transvection, microinjection, cationic lipid-mediated transfection, electroporation, transduction, scrape lading, ballistic introduction or infection.

The desired protein or fragment is then expressed in suitable host cells, such as for example, mammalian cells, yeast, bacteria, or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the desired protein or fragment.

Conventional procedures for isolating recombinant proteins from transformed host cells, such as isolation by initial extraction from cell pellets or from cell culture medium, followed by salting-out, and one or more chromatography steps, including aqueous ion exchange chromatography, size exclusion chromatography steps, high performance liquid chromatography (IIPLC), and affinity chromatography may be used to isolate the recombinant protein or fragment.

Methods of Protecting Against Lethal Infection with B. anthracis Using Peptide-Based Immunogenic Compositions

The present invention also provides methods for eliciting an immune response which protects an animal subject against lethal infection with B. anthracis. The animal subject may be any mammal, including a human subject. In one aspect, the method comprises administering one of the above-described protein or peptide-based immunogenic compositions to the subject. The immune response prophylactically prevents a lethal B. anthracis infection in the animal. The active immunity elicited by immunization with the above-described protein-based immunogenic compositions can prime or boost a cellular or humoral immune response.

The LF protein, PA protein, and fragments thereof can be prepared in admixture with an pharmaceutically acceptable carrier or diluent. Optionally, the LF protein, PA protein, and fragments thereof can be prepared in admixture with an adjuvant. The term “adjuvant” as used herein refers to a compound or mixture which enhances the immune response to an antigen. Adjuvants include, but are not limited to, complete Freund's adjuvant, incomplete Freund's adjuvant, saponin, mineral gels such as aluminum hydroxide, surface active substances such as lysolecithin, pluronic polyols, polyaninons, peptides, oil or hydrocarbon emulsions, keyhole limpet hemocyanins, dinitrophenol, and potentially useful human adjuvants such as BCG (bacille Calmette-Guerin) and Corynebacterium parvum. Selection of an adjuvant depends of the animal subject to be vaccinated. Preferably, a pharmaceutically acceptable adjuvant is used. For example, oils or hydrocarbon emulsion adjuvants should not be used for human. One example of an adjuvant suitable for use with humans is alum (alumina gel.)

Preferably, the protein or peptide-based immunogenic compositions are administered to the animal subject by injection, such as for example intramuscular (i.m.), intradermal (i.d.), intranasal (i.n.) or sub-cutaneous (s.c.) injection. It is contemplated that 2 or more injections over an extended period of time will be optimal. The immunogenic compositions are administered in an dosage sufficient to prevent a lethal B. anthracis infection in a subject through a series of immunization challenge studies using a suitable animal host system, e.g. rhesus macaques which are thought to be an acceptable standard for human use considerations.

Nucleic Acid-Based Immunogenic Composition

In another aspect, the present invention relates to nucleic-acid based immunogenic compositions which comprise a polynucleotide which encodes the B. anthracis LF protein or, preferably, a mutated form of the LF protein, referred to hereinafter as the “LF polynucleotide”, or an immunogenic fragment thereof, referred to hereinafter as the “LF fragment polynucleotide” and methods of using such immunogenic compositions. The LF polynucleotide may encode a full-length mature LF protein or, preferably, a mutated LF protein such as LF7. In one embodiment, the LF polynucleotide comprises the nucleotide sequence, SEQ ID NO. 1, shown in FIG. 1. In another embodiment, the LF polynucleotide comprises nucleotide 100 through 2430 of SEQ ID NO. 1. In one embodiment, the LF fragment polynucleotide comprises nucleotide 125 through nucleotide 855 of the sequence, SEQ ID NO. 1, shown in FIG. 1. The LF polynucleotide or LF fragment polynucleotide is operably linked to a promoter which drives expression of the protein or fragment. Such promoter may be a constitutive promoter, such as for example the viral promter derived from cyomegalovirus (CMV) Alternatively, the promoter may be an inducible promoter such as, for example, the lac promoter or a tissue specific promoter, such as the whey acidic protein promoter.

In another aspect, the present invention relates to immunogenic compositions which comprise an LF polynucleotide which encodes a mutated LF protein or LF fragment polynucleotide and a polynucleotide which encodes the B. anthracis PA protein, referred to hereinafter as the “PA polynucleotide”, or an immunogenic fragment thereof, referred to hereinafter as the “PA fragment polynucleotide”. The PA polynucleotide may encode a full-length mature PA protein or, alternatively, a full-length, immature PA protein which comprises a nucleotide sequence encoding a signal sequence. In one embodiment, the PA polynucleotide comprises the nucleotide sequence, SEQ ID NO. 3, shown in FIG. 2. In one embodiment, the PA fragment polynucleotide comprises nucleotide 88 through nucleotide 2295 of the sequence, SEQ ID NO. 3, shown in FIG. 2. The PA polynucleotide and PA fragment polynucleotide are operably linked to a promoter which drives expression of the PA protein or fragment thereof.

The polynucleotide may be either a DNA or RNA sequence. All forms of DNA, whether replicating or non-replicating, which do not become integrated into the genome, and which are expressible, are within the methods contemplated by the invention. When the polynucleotide is DNA, it can also be a DNA sequence which is itself non-replicating, but is inserted into a plasmid, and the plasmid further comprises a replicator. The DNA may be a sequence engineered so as not to integrate into the host cell genome. The polynucleotide sequences may code for a polypeptide which is either contained within the cells or secreted therefrom, or may comprise a sequence which directs the secretion of the peptide. With the availability of automated nucleic acid synthesis equipment, both DNA and RNA can be synthesized directly when the nucleotide sequence is known or by methods which employ PCR cloning.

The LF polynucleotide, LF fragment polynucleotide, PA polynucleotide, and PA fragment polynucleotides can be incorporated into the immunogenic compositions in one of several forms including a linear molecule, a plasmid, a viral construct, or a bacterial construct, such as for example a Salmonella construct to provide a vaccine. In those cases where the immune response is elicited by administration of both the LF polynucleotide or LF fragment polynucleotide and the PA polynucleotide or PA fragment polynucleotide, the polynucleotides may be incorporated into separate nucleic acid molecules which are co-administered to the subject. Alternatively, the LF polynucleotide (or LF fragment polynucleotide) and PA polynucleotide (or PA fragment polynucleotide) may be incorporated into the same nucleic acid. In such case, the mutated LF polynucleotide and PA polynucleotide may be operably linked to separate promoters or to the same promoter.

The present invention also relates to methods of using the nucleic acid-based immunogenic compositions to elicit a protective immune response against lethal infection with B. anthracis in an animal subject. The method comprises administering one of the above-described nucleic acid-based immunogenic compositions to the subject. The nucleic acid-based compositions are administered at a dosage sufficient to elicit, prime, or boost an immune response which prophylactically protects against a lethal B. anthracis infection in the animal. The nucleic acid-based immunogenic compositions are, preferably, incorporated into vaccines which are administered to the animal subject.

Viral Vaccines

Various genetically engineered virus hosts, i.e. recombinant viruses, can be use to prepare LF and PA vaccines which comprise the present immunogenic compositions. Examples of recombinant virus host which can be used to prepare such vaccines include, but are not limited to vaccinia virus, recombinant canarypox, and defective adenovirus. Other suitable viral vectors include retroviruses that are packaged in cells with amphotropic host range and attenuated or defective DNA virus, such as herpes simplex virus, papillomavirus, Epstein Barr virus, and adeno-associated virus.

Nucleic Acid Vaccine

In a preferred embodiment, the method comprises directly administering a nucleic acid, particularly a DNA, which encodes the desired protein or proteins or fragments thereof, into the subject. Such compositions which are termed herein “nucleic acid based vaccines” or DNA vaccines are described in U.S. Pat. No. 5,589,466 which issued in December, 1996 to Felgner et al, the disclosure of which is hereby incorporated by reference in its entirety. Introducing DNA that encodes the LF protein or fragment thereof, alone or in combination with a DNA that encodes the PA protein or a fragment thereof, induces both cell-mediated and humoral responses. The advantages of this approach, i.e, using a DNA vaccine which encodes the mutated LF protein or fragment thereof, alone or in combination with a DNA encoding the PA protein or a fragment thereof, are as follows:

1).Both components (humoral and cell-mediated) of the immune system are stimulated, which results in longer term immune memory response.

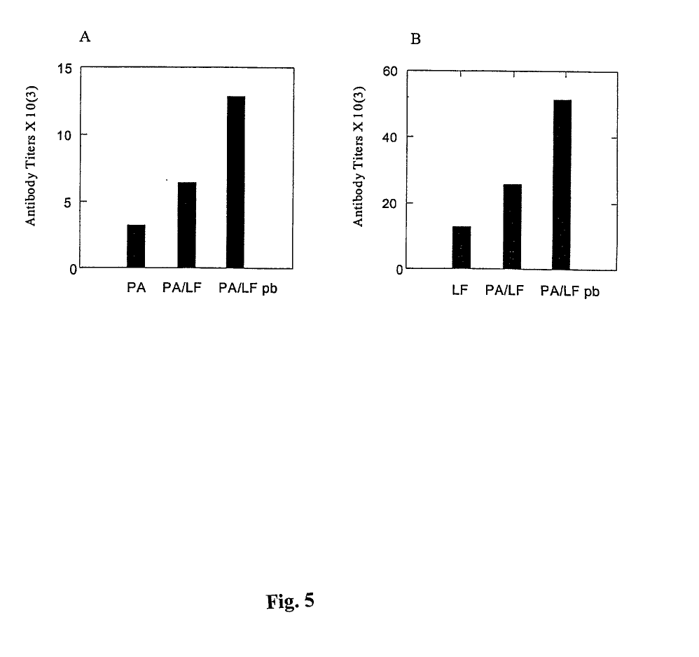

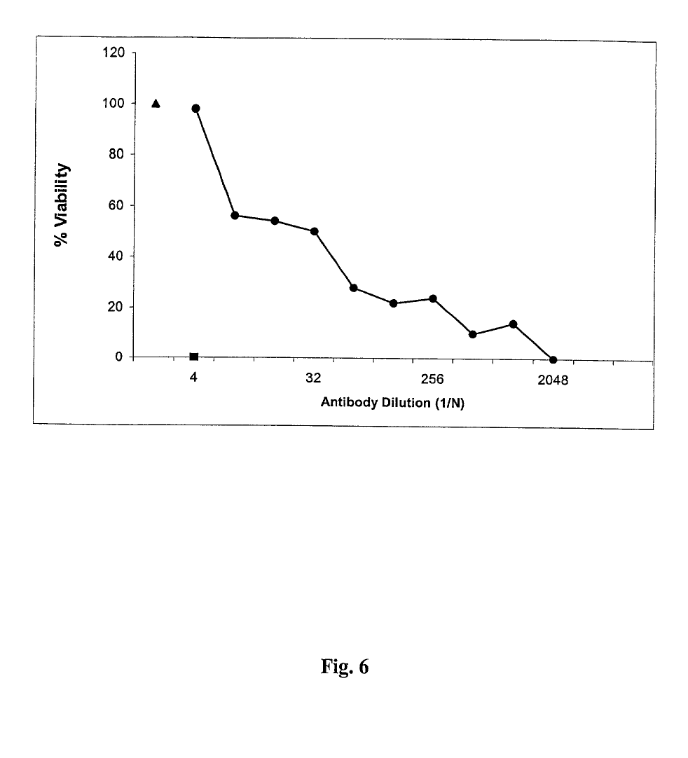

2). The combined use of a mutated gene LF and PA gene or their fragments results in a higher level of immune response, as judged by overall serum antibody titers for the LF and PA antigens, than the use of either LF or PA genes in separate immunizations; i.e. there is a synergistic effect when both genes/proteins are used together in an immunization (see FIG. 5).

3). DNA-based formulations for immunization are less expensive to produce, store and administer since they do not require the expression and/or purification of proteins.

4). DNA-based formulations for immunization contain fewer possible components to contribute to side effects (i.e. contaminants such as endotoxin or other proteins).

5). DNA-based formulations for immunization can be made highly specific and are easily manipulated at the genetic level to effect changes or modify the original composition for improvement of the immune response

6). DNA-based formulations are readily amenable to a variety of delivery mechanisms thus constituting a more versatile immunogenic system.

In preferred embodiments, the nucleic acid-based composition is introduced into muscle tissue; in other embodiments the nucleic acid-based composition is incorporated into tissues of skin, brain, lung, liver, spleen or blood. The preparation may be injected into the animal subject by a variety of routes, which may be intradermally, subdermally, intrathecally, or intravenously, or it may be placed within cavities of the body. In a preferred embodiment, the nucleic acid-based composition is injected intramuscularly. In still other embodiments, the nucleic acid based-composition is impressed into the skin or administered by inhalation.

It is contemplated that the nucleic acid based compositions will be administered to the animal subject 2 or more times over an extended period of time will be optimal. The nucleic acid-based immunogenic compositions are administered in an dosage sufficient to prevent a lethal B. anthracis infection in the subject.

The dosage to be administered depends on the size of the subject being treated as well as the frequency of administration and route of administration. Ultimately, the dosage will be determined using clinical trials. Initially, the clinician will administer doses that have been derived from animal studies.

The following examples are for illustration only and are not intended to limit the scope of the invention.

### EXAMPLE 1

Inducing a Protective Immune Response Against Challenge with B. antracis Toxin by Adminstration of a DNA Plasmid Comprising an Immunogenic Fragment of LF Alone

A. Materials and Methods

The eucaryotic expression plasmid pCI (Promega, Inc.) was used to prepare a construct for the expression of a truncated version of the LF protein. The plasmid construct pCLF4 encodes the LF protein fragment consisting of amino acids 9-252 which includes the PA binding site. This plasmid was constructed from a PCR-amplified fragment using the primers 5′-CTGAAACCATCACGTAAAA-3′ and 3′-AGCAAGAAATAAATCTATAGTCTAGA-5′ which contain Xba cut sites. The Xba-digested PCR and pCI plasmid fragments were ligated to form the pCLF4 plasmid used in these studies. The resulting plasmid construct pCLF4 does not contain a signal sequence for secretion of the expressed gene product. All plasmids were purified from E. coli DH5α using the Endo-free plasmid preparation kits (Qiagen) and resuspended in PBS before use.

Protein preparations. The LF and LF7 antigens used in these studies were expressed and purified as previously described (Leppla 1988; Park 2000. Optimized production and purification of Bacillus anthracis lethal factor. Prot. Exp. Purif 18:293-302). LF7 is the full-length LF protein which contains a mutation at position 687 (E687C) in the zinc-binding active site thus eliminating the metalloproteinase activity of LF.

DNA Vaccination

Purified plasmid DNA was coated onto 1 micron gold particles according to the manufacturer's instructions (BioRad Laboratories, Richmond, Calif.). Separate groups of female BALB/c mice at 4-5 weeks in age (Jackson Laboratories Bar Harbor, Me.) were immunized (i.d.) in the abdomen via biolistic particle injection (Bio-Rad Helios Gene Gun, Richmond, Calif.) on days 0, 14, and 28 with approximately 1 ug of plasmid DNA coated onto gold particles for each injection For the prime-boost immunization experiments, separate groups of BALB/c mice were first immunized twice with plasmid DNA as described above followed by a third and final protein boost of purified antigen resuspended in Freund's incomplete adjuvant (1:1 ratio of adjuvant to protein, v/v). The protein immunizations were administered i.m. Blood samples were obtained two weeks following each vaccination and the sera was pooled and stored at −20° C. until analyzed.

Mouse Macrophage Protection Assay

The cytotoxicity of the purified lethal toxin was established using a previously described macrophage cytotoxicity assay (Varughese 1998; Park 2000). For the protection assay J774A.1 mouse macrophage cells were placed in flat-bottomed 96-well microtiter plates at a concentration of 6×10⁴ cells/well in Dulbecco's modified Eagle's medium (DMEM) (Sigma) with 7% fetal bovine serum, 4.5 g/L glucose, and 2 mM L-glutamine and incubated for 24 hrs at 37° C. Serum from a pCLF4 immunized New Zealand White rabbit was serially diluted and incubated with LF protein for 1 hour to allow neutralization to occur. Following this incubation, the LF-anti-LF4 mixture was added to PA protein to achieve a final concentration of 3 ug/ml lethal toxin (Letx). This preparation was incubated at room temperature for 1 hour prior to being added to the cells, which were then incubated for an additional 7 hrs 37° C. At the end of the incubation, 100 ul/well of 0.5mg/ml MTT (3-[4,5-dimethylthiazol-2-yl]-2,5-diphenyltetrazolium bromide) (Sigma) was added followed by a 1 hour incubation. Cells which survive exposure to lethal toxin are able to oxidize MTT to an insoluble purple pigment thus providing a proportional measure of the viability of the cells. At the end of the incubation period the culture supernatant fraction was aspirated and 50 ul of 0.5% (w/v) SDS, 25 mM HCl in 90% (v/v) 2-propanol was added and the suspension was vortexed. The A450 was determined using a microplate reader (Bio-Tek Instruments, Inc.).

In vivo Protection Assay

PA and LF were purified from B. anthracis as previously described (Leppla 1988, Production and purification of anthrax toxin, p. 103-116 In S. Harshman (ed.), Methods in Enzymology. Academic Press, Inc., Orlando, Fla.). Plasmid-immunized BALB/c mice which had received a total of three injections were challenged with purified lethal toxin two weeks following the third and final injection. The challenge was conducted by tail vein injection of a previously mixed combination of purified PA and LF proteins (60 ug PA and 25-30 ug LF per mouse) which is equivalent to approximately five x LD₅₀ of lethal toxin.

ELISA Assay for Anti-LF Antibodies

Antibody titers against the LF determined by ELISA assay. Briefly, Immulon 4 96-well plates (Dynatech Laboratories, Inc., Chantilly, Va.) were coated with 100 ng of purified PA or LF7 protein dissolved in 0.1 M carbonate buffer, pH 9.6 at 4° C. overnight. Plates were washed with PBS (phosphate buffered saline, 0.15 M phosphate buffer, pH 7.3) and blocked 1% BSA in TBS (Tris-buffered saline, pH 7.3). Serum samples were serially diluted in TBS 0.05% Tween-20 and added to the plates. All incubations were carried out at 37° C. for one hour. Anti-mouse IgG conjugated to horseradish peroxidase (Amersham Life Science, Arlington Hts., Ill.) was added as a secondary antibody. The presence of bound antibody was detected following a 30 min incubation in the presence of ABTS substrate (Zymed, S. San Francisco, Calif.) and absorbance was read at 405 nm using a Bio-Rad Model 550 plate reader. Antibody titers were defined as the highest serum dilution that results in an absorbance value two times greater than a non-immune serum control with a minimum value of 0.05. Antibody isotypes were determined in a similar manner, except anti-mouse IgG₁ or anti-mouse IgG₂ₐ conjugated to alkaline phosphatase was used as the secondary antibody (Zymed Laboratories, San Francisco, Calif., USA). Antibody quantitation was determined by ELISA analysis using a standard curve with purified IgGi and IgG₂ antibody reagents.

### EXAMPLE 2

Inducing a Protective Immune Response Against Challenge with B. anthracis Toxin by Co-Adminstration of a DNA plasmid Encoding an Immunogenic Fragment of LF and DNA Plasmid Encoding an Immunogenic Fragment of PA

Materials and Methods

The eucaryotic expression plasmid pCI (Promega, Inc.) was used to prepare a construct for the expression of a truncated version of the LF protein. The gene fragment encoding amino acids 175-735 of the PA protein was PCR amplified using the plus strand primer (5′-CTCGAGACCATGGTT-3′) and minus strand primer (3′-TAAGGTAATTCTAGA-5′) using pYS2 as a template (Welkos 1988; Singh 1994). Included in the primer sequences are Xho and Xba restriction cut sites, respectively. The PA gene fragment expressed in these studies represents the PA₆₃ protease-cleaved fragment of the full-length 83 kDa protein that is active in vivo (Gordon 1995). The PCR reaction product was digested with Xhol and Xba and ligated into the pCI vector which had been cut with the same two restriction enzymes.

DNA vaccination of animals was performed as described above in Example 1. Immunization groups included the pCPA, pCLF4, a 1:1 mixture of the pCPA and pCLF4 plasmids and the pCI plasmid as a vector control. (Leppla 1988). Plasmid-immunized BALB/c mice which had received a total of three injections were challenged with purified lethal toxin two weeks following the third and final injection. The challenge was conducted by tail vein injection of a previously mixed combination of purified PA and LF proteins (60 ug PA and 25-30 ug LF per mouse) which is equivalent to approximately five×LD₅₀ of lethal toxin. Antibody titers against PA were determined as described above in Example 1.

Results

Immunization with Plasmids Encoding PA and/or LF

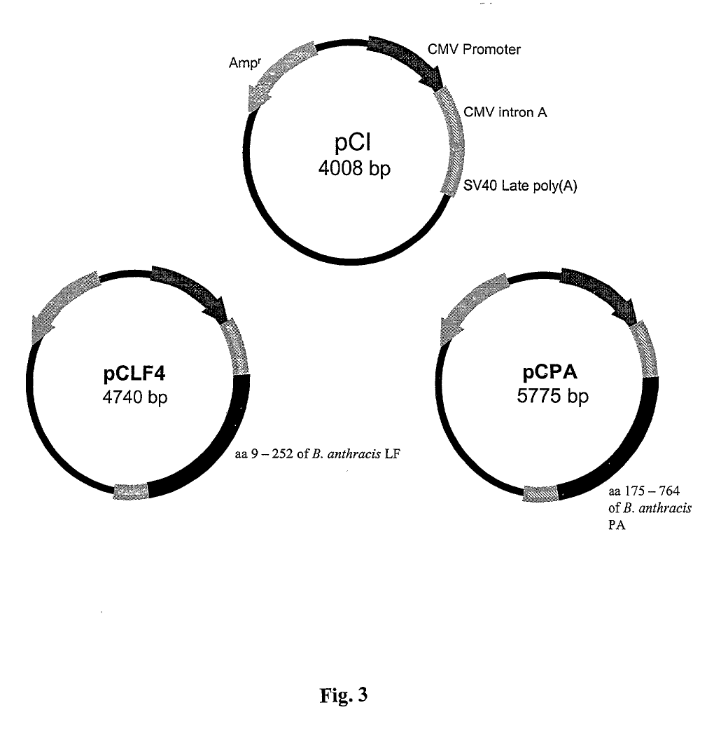

These examples utilized the pCI mammalian expression vector (Promega) which utilizes the human cytomegalovirus (CMV) immediate-early enhancer-promoter region for strong, constitutive expression of the incorporated gene (FIG. 3). Use of this expression vector results in high level expression of a non-secreted form of the encoded gene product. In these examples we chose to express only partial sequences of the PA and LF genes as shown in FIG. 3. The pCPA plasmid expresses a truncated version of the PA gene product (aa 175-735) which is the PA₆₃ antigen lacking the furin cleavage site (aa164-167) yet is fully functional in vivo (Gordon 1995. Proteolytic activation of bacterial toxins by eukaryotic cells is performed by furin and by additional cellular proteases, Infect. Immun. 63:82-87.). The pCLF4 plasmid expresses a truncated form of LF (aa 9-252) which lacks the catalytic domain of LF, yet retains PA₆₃ binding activity and is therefore capable of interacting with the truncated form of PA expressed from pCPA (Arora, Klimpel et al. 1992. Fusions of anthrax toxin lethal factor to the ADP-ribosylation domain of Pseudomonas exotoxin A are potent cytotoxins which are translocated to the cytosol of mammalian cells. J Biol Chem 267(22):15542-8.).

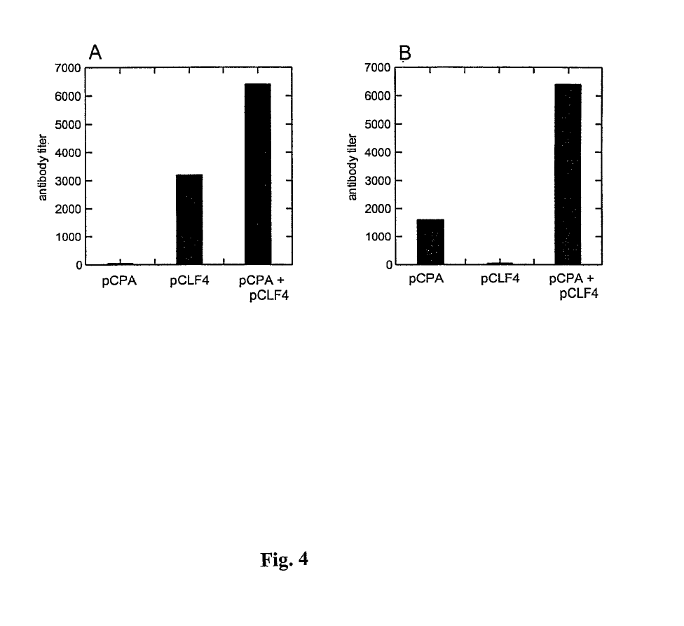

Groups of female BALB/c mice were administered plasmid DNA (PCPA, pCLF4, or pCI) which had previously been coated onto 1 micron gold beads according to the manufacturer's instructions (BioRad Laboratories, Richmond, Calif.) and introduced via biolistic particle injection (gene gun). Each injection introduced approximately 1 ug of plasmid DNA. Injections were given at two week intervals for a total of three injections. Separate groups of mice received plasmid injections of pCPA, pCLF4, a 1:1 mixture of these two plasmids, or a vector control consisting of the pCI plasmid. Two weeks following the third and final injection, pooled antisera was evaluated for antibody response against the PA and/or LF antigens. FIG. 4 demonstrates that collectively each immunized group produced significant antibody titers against the antigen to which they had been respectively immunized. Significantly, antibody titers at day 42 against the LF antigen following DNA immunization appear to be about twice the level of antibody titers against the PA antigen observed following pCPA immunization, suggesting that the LF antigen may induce a higher antibody response due to the increased immunogenicity of the LF protein. It is also to be noted that co-immunization with the pCPA and pCLF4 plasmids resulted in a significantly higher overall antibody response against either the PA or LF antigens when compared to the antibody response following separate immunization with either gene alone. This result suggests the possibility of some form of synergistic effect when these two genes are co-administered. This observation is also supported by the results of a second series of pCPA and pCLF4 immunizations in a separate group of BALB/c mice (FIG. 5). These results demonstrate that significantly higher endpoint titers against both the PA and LF antigens are obtained when mice are co-immunized with both the PA and LF genes.

Plasmid Immunization Results in a Protective Response

To determine whether DNA-based immunization alone can provide protection against exposure to the lethal toxin (Letx), small groups of BALB/c mice which had been immunized three times with plasmids pCPA, pCLF4, a 1:1 combination of pCPA and pCLF4, or the plasmid vector (pCI), were challenged with a 5×LD₅₀ dose of Letx administered intravenously. The results of this challenge study are presented in Table 1 below where it can be seen that all animals plasmid-immunized against either PA or LF survived. Control mice succumbed to the lethal toxin challenge within hours. These results demonstrate that DNA-based immunization alone can provide a protective response against exposure to the lethal anthrax toxin.

Table 1. Vaccination with plasmids pCPA, pCLF4, or a combination of them confers protection against lethal anthrax toxin challenge.

| | | Immunized Mice | | | |
|---|---|---|---|---|---|
| Challenge Dose | LD₅₀ | Vector | pPA | pLF4 | pLF4 + pPA |
| 60 ug PA, 25 ug LF4 | 5 | 0/3 | 3/3 | 3/3 | 4/4 |

A mixture of PA (60 ug/mouse) and LF (25 ug/mouse) was injected i.v. into multiply immunized or vector treated BALB/c mice. Values shown are number of survivors/number challenged.

Comparison Between Prime/boost and DNA-only Immunization

The ability of the prime/boost method and the DNA-only immunization to enhance antibody titers against the PA and LF antigens were compared. The prime boost method involves priming the immune system with a series of three plasmid-based immunizations followed by a final booster immunization with the protein antigen. In FIG. 5 it can be seen that co-administration of the pCPA and pCLF4 plasmids followed by a final protein booster immunization with the rPA and rLF7 antigens produces a substantially higher endpoint titer against either the PA or LF antigens at the same timepoint when compared to antibody titers resulting from DNA-based immunization alone. It is also observed that there is a consistently higher antibody titer formed against the LF antigen regardless of the immunization regimen used.

Antibody Type

Further analysis of the antisera from plasmid immunized mice indicates that the predominant antibody type produced as a result of these immunizations is of the IgG₁ subclass (Table 2), although it is noted that significant levels of IgG₂ subclass antibodies are also produced. Importantly, protection against anthrax toxin has been associated with the production of IgG1 class antibodies or a T_{H}2 class of response. Thus while the majority antibody response is characteristic of a T_{H}2 type immune response, it is clear that there is also a significant T_{H}1 type response as well. These results are consistent with the previous report by Gu et al (Gu 1999. Protection against anthrax toxin by vaccination with a DNA plasmid encoding anthrax protective antigen. Vaccine 17:340-344.).

TABLE 2
IgG1 and IgG2a antibody levels (ug/ml) against purified mutant lethal
factor and protective antigen proteins.

| | anti-PA | | anti-LF | |
|---|---|---|---|---|
| | IgG1 | IgG2a | IgG1 | IgG2a |
| PA^{a} | 0.6 | 0.5 | — | — |
| LF^{b} | — | — | 38 | 0.2 |
| LF/PA^{c} | 0.3 | 0.1 | 69 | 0.1 |
| PA prime boost^{d} | 2 | 0.1 | — | — |
| LF prime boost^{e} | — | — | 1164 | 2.7 |
| PA/LF prime boost^{f} | 13 | 4 | 538 | 2.5 |

^{a}serum collected from mice immunized with a DNA vaccine encoding PA
^{b}serum collected from mice immunized with a DNA vaccine encoding LF
^{c}serum collected from mice immunized with a DNA vaccine encoding PA and LF
^{d}serum collected from mice immunized with a DNA vaccine encoding PA and boosted with 12.5:g of purified PA protein
^{e}serum collected from mice immunized with a DNA vaccine encoding LF and boosted with 12.5:g of purified LF protein
^{f}serum collected from mice immunized with a DNA vaccine encoding PA and LF and boosted with 12.5:g of purified PA and LF protein

### EXAMPLE 3

Inducing a Protective Immune Response Against Challenge with B. anthracis Sores by a Prime Boost Method Which Employs a DNA Plasmid Encoding an Immunogenic Fragment of LF, a DNA Plasmid Encoding an Immunogenic Fragment of PA, and and a Booster Immunization with Purified rPA/rLF7

Female A/J mice were immunized with 1 ug plasmid in PBS via gene gun three times at 2 week intervals and received a final protein boost (20 ug i.m. in incomplete Freund's adjuvant). Two weeks following the protein boost all animal were injected i.p with the 1×10⁵ or 1×10⁶ viable Sterne strain spores and observed for a period of 14 days. As shown in Table 3 below, controls succumb within 72 hours; survivors were determined at 14 days.

TABLE 3
Prime-boost vaccination study in A/J mouse i.p spore challenge model

| | | Survivors/challenged mice | | |
|---|---|---|---|---|
| Challenge Dose | LD₅₀ | Vector | pCPA | pCPA + pCLF4 |
| 1 × 10⁵ spores | 100× | 0/5 | 5/5 | 5/5 |
| 1 × 10⁶ spores | 1000× | 0/5 | 4/5 | 5/5 |

Although the invention has been described with regard to a number of preferred embodiments, which constitute the best mode presently known to the inventors for carrying out this invention, it should be understood that various changes and modifications as would be obvious to one having the ordinary skill in this art may be made without departing from the scope of the invention which is defined by the claims which are appended hereto.

What is claimed is:

1. A method for protecting an animal subject against lethal infection with B. anthracis, comprising:
administering an immunogenic composition which comprises purified or recombinant B. anthracis lethal factor (LF) protein or an immunogenic fragment thereof to the subject.

2. The method of claim 1 wherein the immunogenic composition comprises a mutated LF protein or an immunogenic fragment of an LF protein, and
further comprising administering an immunogenic composition which comprises purified or recombinant B. anthracis protective antigen(PA) protein or an immunogenic fragment thereof to the subject.

3. The method of claim 1 wherein the LF protein comprises a sequence which is at least 90% identical to a sequence extending from amino acid 1 through amino acid 775 of the sequence set forth in SEQ ID NO:2.

4. The method of claim 1 wherein the LF protein fragment comprises a sequence which is at least 90% identical to a sequence extending from amino acid 9 through amino acid 252 of the sequence set forth in SEQ ID NO. 2.

5. The method of claim 2 wherein the PA protein comprises a sequence which is at least 90% identical to a sequence extending from amino acid 1 through amino acid 735 of the sequence set forth in SEQ ID NO. 4.

6. The method of claim 2 wherein the PA protein fragment comprises a sequence which is at least 90% identical to a sequence extending from amino acid 175 through amino acid 735 of the sequence set forth in SEQ ID NO. 4.

7. A method for protecting a susceptible animal subject against lethal infection with B. anthracis, comprising:
administering a nucleic acid-based immunogenic composition which comprises an isolated polynucleotide which encodes a mutated B. anthracis lethal factor (LF) protein or an immunogenic fragment thereof to the subject, said polynucleotide being operably linked to a promoter which drives expression of the mutated LF protein or the immunogenic LF protein fragment.

8. The method of claim 7 further comprising administering an immunogenic composition which comprises an isolated polynucleotide which encodes B. anthracis protective antigen(PA) protein or an immunogenic fragment thereof to the subject, said polynucleotide being operably linked to a promoter which drives expression of the PA protein or immunogenic fragment thereof in the subject.

9. The method of claim 7 wherein the LF protein comprises a sequence which is at least 90% identical to a sequence extending from amino acid 1 through amino acid 775 of the sequence set forth in SEQ ID NO:2.

10. The method of claim 7 wherein the LF protein fragment comprises a sequence which is at least 90% identical to a sequence extending from amino acid 9 through amino acid 252 of the sequence set froth in SEQ ID NO. 2.

11. The method of claim 8 wherein the PA protein comprises a sequence which is at least 90% identical to a sequence extending from amino acid 1 through amino acid 735 of the sequence set forth in SEQ ID NO. 4.

12. The method of claim 8 wherein the PA protein fragment comprises a sequence which is at least 90% identical to a sequence extending from amino acid 175 through amino acid 735 of the sequence set forth in SEQ ID NO. 4.

13. The method of claim 7 wherein the polynucleotide is a component of a nucleic acid-based vaccine

14. The method of claim 7 wherein the polynucleotide is a component of a viral vaccine.

15. The method of claim 8 wherein administration of the LF polynucleotide and the PA polynucleotide enhance production of antibodies to LF and PA protein in the subject.

16. The method of claim 8 further comprising administering a peptide-based immunogenic composition to the subject, said second immunogenic composition comprising an immunogen selected from the group consisting of a mutated LF protein, an immunogenic fragment of an LF protein, a PA protein, an immunogenic fragment of a PA protein, and combinations thereof, wherein said second immunogenic composition is administered to the subject before or after administration of the polynucleotide-based immunogenic composition.

17. An immunogenic composition for preparing a vaccine which protects a subject against lethal infection B. anthracis, said immunogenic composition comprising a purified or recombinant lethal factor (LF) protein or immunogenic fragment thereof and a pharmaceutically acceptable carrier or diluent.

18. The immunogenic composition of claim 17 wherein said immunogenic composition comprises a mutated LF protein or an immunogenic fragment of an LF protein and a purified or recombinant B. anthracis PA protein or immmunogenic fragment thereof.

19. The immmunogenic composition of claim 18
wherein the mutated LF protein comprises a sequence which is at least 90% identical to a sequence extending from amino acid 1 through amino acid 735 of the sequence set forth in SEQ ID NO:2.

20. The immunogenic composition of claim 18 wherein the LF protein fragment comprises a sequence which is at least 90% identical to a sequence extending from amino acid 9 through amino acid 252 of the sequence set froth in SEQ ID NO. 2.

22. The immunogenic composition of claim 18 wherein the PA protein comprises a sequence which is at least 90% identical to a sequence extending from amino acid 1 through amino acid 735 of the sequence set forth in SEQ ID NO. 4.

22. The immunogenic composition of claim 18 wherein the PA protein fragment comprises a sequence which is at least 90% identical to a sequence extending from amino acid 175 through amino acid 735 of the sequence set forth in SEQ ID NO. 4.

23. A nucleic-acid based immunogenic composition for preparing a vaccine which protects a subject against lethal infection B. anthracis, said immunogenic composition comprising a polynucleotide which encodes a mutated lethal factor (LF) protein or immunogenic fragment of LF protein and a pharmaceutically acceptable carrier or diluent.

24. The immunogenic composition of claim 23 further comprising an isolated polynucleotide which encodes B. anthracis protective antigen(PA) protein or an immunogenic fragment thereof to the subject, said polynucleotide being operably linked to a promoter which drives expression of the PA protein,

25. The immunogenic composition of claim 24 wherein the PA polynucleotide comprises a sequence comprising consecutively nucleotide 610 through nucleotide 2295 of the sequence set forth in SEQ ID NO. 3.

26. The immunogenic composition of claim 24 wherein the LF polynucleotide and the PA polynucleotide are on separate DNA constructs.

27. The immunogenic composition of claim 24 wherein the LF polynucleotide and the PA polynucleotide are on the same DNA construct.

28. A method for inducing an immune response which inactivates the B. antracis toxin in an animal, said method comprising administering to the animal an immunogenic composition which comprises an isolated nucleic acid which encodes a mutated B. anthracis lethal factor (LF) protein or an immunogenic fragment of LF protein to the subject, said nucleic acid being operably linked to a promoter which drives expression of the mutated LF protein or the immunogenic LF protein fragment.

29. The method of claim 26 further comprising administering an immunogenic composition which comprises an isolated nucleic acid which encodes B. anthracis protective antigen(PA) protein or an immunogenic fragment thereof to the subject, said nucleic acid being operably linked to a promoter which drives expression of the PA protein or immunogenic fragment thereof in the subject.

30. The method of claim 28 wherein the method protects the subject from challenge with a dose which is at least 3 times the LD₅₀ of the lethal toxin.


SEQUENCE LISTING (8 sequences)

SEQ ID NO: 1 - DNA, 2430 bp, Bacillus anthracis
  CDS (1)..(2430)
atgaatataaaaaaagaatttataaaagtaattagtatgtcatgtttagtaacagcaattactttgagtggtcccgtctttatcccccttgtacagggggcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccttggaagaacttaaagatcaacggatgctgtcaagatatgaaaaatgggaaaagataaaacagcactatcaacactggagcgattctttatctgaagaaggaagaggacttttaaaaaagctgcagattcctattgagccaaagaaagatgacataattcattctttatctcaagaagaaaaagagcttctaaaaagaatacaaattgatagtagtgattttttatctactgaggaaaaagagtttttaaaaaagctacaaattgatattcgtgattctttatctgaagaagaaaaagagcttttaaatagaatacaggtggatagtagtaatcctttatctgaaaaagaaaaagagtttttaaaaaagctgaaacttgatattcaaccatatgatattaatcaaaggttgcaagatacaggagggttaattgatagtccgtcaattaatcttgatgtaagaaagcagtataaaagggatattcaaaatattgatgctttattacatcaatccattggaagtaccttgtacaataaaatttatttgtatgaaaatatgaatatcaataaccttacagcaaccctaggtgcggatttagttgattccactgataatactaaaattaatagaggtattttcaatgaattcaaaaaaaatttcaaatatagtatttctagtaactatatgattgttgatataaatgaaaggcctgcattagataatgagcgtttgaaatggagaatccaattatcaccagatactcgagcaggatatttagaaaatggaaagcttatattacaaagaaacatcggtctggaaataaaggatgtacaaataattaagcaatccgaaaaagaatatataaggattgatgcgaaagtagtgccaaagagtaaaatagatacaaaaattcaagaagcacagttaaatataaatcaggaatggaataaagcattagggttaccaaaatatacaaagcttattacattcaacgtgcataatagatatgcatccaatattgtagaaagtgcttatttaatattgaatgaatggaaaaataatattcaaagtgatcttataaaaaaggtaacaaattacttagttgatggtaatggaagatttgtttttaccgatattactctccctaatatagctgaacaatatacacatcaagatgagatatatgagcaagttcattcaaaagggttatatgttccagaatcccgttctatattactccatggaccttcaaaaggtgtagaattaaggaatgatagtgagggttttatacacgaatttggacatgctgtggatgattatgctggatatctattagataagaaccaatctgatttagttacaaattctaaaaaattcattgatatttttaaggaagaagggagtaatttaacttcgtatgggagaacaaatgaagcggaattttttgcagaagcctttaggttaatgcattctacggaccatgctgaacgtttaaaagttcaaaaaaatgctccgaaaactttccaatttattaacgatcagattaagttcattattaactcataa

SEQ ID NO: 2 - protein, 809 aa, Bacillus anthracis
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 3 - DNA, 2295 bp, Bacillus anthracis
  CDS (1)..(2295)
atgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtgaaacgagaacaataagtaaaaatacttctacaagtaggacacatactagtgaagtacatggaaatgcagaagtgcatgcgaatacttctacaagtaggacacatactagtgaagtacatggaaatgcagaagtgcatgcggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataa

SEQ ID NO: 4 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHANTSTSRTHTSEVHGNAEVHAVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 5 - DNA, 19 bp, synthetic construct
ctgaaaccatcacgtaaaa

SEQ ID NO: 6 - DNA, 26 bp, synthetic construct
agcaagaaataaatctatagtctaga

SEQ ID NO: 7 - DNA, 15 bp, synthetic construct
ctcgagaccatggtt

SEQ ID NO: 8 - DNA, 15 bp, synthetic construct
taaggtaattctaga